(19) United States
(12) Patent Application Publication
(10) Pub. No.: US 2022/0296847 A1
(43) Pub. Date: Sep. 22, 2022

(54) WEARABLE DEVICE OPERABLE TO DETECT AND/OR MANAGE USER STRESS

(71) Applicant: HAPPY HEALTH, INC., Austin, TX (US)

(72) Inventors: Dustin M. FRECKLETON, Austin, TX (US); Byron P. OLSON, Boone, IA (US); Nithin O. RAJAN, Austin, TX (US); David E. CLIFT-REAVES, Austin, TX (US)

(73) Assignee: HAPPY HEALTH, INC., Austin, TX (US)

(21) Appl. No.: 17/619,650

(22) PCT Filed: Jun. 17, 2020
(86) PCT No.: PCT/US2020/038236

§ 371 Date: Dec. 16, 2021

Related U.S. Application Data
(60) Provisional application No. 62/862,420, filed on Jun. 17, 2019.

(51) Int. Cl. A61M 21/00

(52) U.S. Cl. CPC A61M 21/00; A61M 2021/0088; A61M 2230/04; A61M 2230/65; A61M 2230/20; A61M 2230/42; A61M 2230/50

(57) ABSTRACT

Stress management apparatus includes a wearable device having one or more physiological sensors operable to be engaged with a body of a user. One or more processors communicatively coupled with the wearable device having a memory storing instructions when executed operable to: detect one or more physiological indicators of stress; suggest a stress intervention to the user; monitor compliance with the stress intervention; and track a reduction of the one or more physiological indicators of stress.

### CROSS-REFERENCE TO RELATED APPLICATIONS

This application claims the benefit of U.S. Provisional Application No. 62/862,420, filed on Jun. 17, 2019, the contents of which are incorporated by reference herein in its entirety.

### FIELD

The present inventive concept relates generally a wearable device operable to detect physiological measurements.

### BACKGROUND

Wearable devices are prominent in society and provide users with multiple data points regarding their physiological status.

### BRIEF DESCRIPTION OF THE DRAWINGS

The novel features of the disclosure are set forth with particularity in the appended claims. A better understanding of the features and advantages of the present inventive concept will be obtained by reference to the following detailed description that sets forth illustrative examples, in which the principles of the disclosure are utilized, and the accompanying drawings of which:

FIG. 1 is a diagrammatic view of a wearable device, according to at least one instance of the present disclosure;

FIG. 2A is a diagrammatic view of a wearable device, according to at least one instance of the present disclosure;

FIG. 2B is a diagrammatic sectional view of a wearable device, according to at least one instance of the present disclosure;

FIG. 2C is a diagrammatic view of a spatially-resolved near-infrared spectroscopy (NIRS) sensor of a wearable device, according to at least one instance of the present disclosure;

FIG. 3 is a block diagram of a wearable device, according to at least one instance of the present disclosure;

FIG. 4 is a diagrammatic view of a wearable device system, according to at least one instance of the present disclosure;

FIG. 5. is a block diagram of a stress management system, according to at least one instance of the present disclosure;

FIG. 6 is a flowchart of stress management system operable with the wearable device system, according to at least one instance of the present disclosure;

FIG. 7 is a diagrammatic representation of a physiological response to stress including skin temperature, ambient temperature, activity, and pulse perfusion, according to at least one instance of the present disclosure;

FIG. 8 is a diagrammatic representation of a physiological response to stress including palmer electrodermal activity (EDA), finger EDA, and heart rate, according to at least one instance of the present disclosure;

FIG. 9A is a flowchart of a breathing intervention exercise, according to at least one instance of the present disclosure;

FIG. 9B is a user respiration rate during a breathing intervention exercise, according to at least one instance of the present disclosure;

FIG. 9C is a user respiration rate during a breathing intervention exercise, according to at least one instance of the present disclosure;

FIG. 9D is a data plot of a respiration rate in view of a stress index measured by a wearable device, according to at least one instance of the present disclosure;

FIGS. 10A, B, C are a data plot of a deep self-paced breathing signal, frequency modulation of heart rate caused by lung inflation, and a frequency content of the frequency modulation, according to at least one instance of the present disclosure;

FIGS. 10D, E, F are a data plot of a box breathing signal, frequency modulation of heart rate caused by lung inflation, and a frequency content of the frequency modulation, according to at least one instance of the present disclosure;

FIG. 11A is a data plot of a stress indicator level of a first user, according to at least one instance of the present disclosure;

FIG. 11B is a data plot of a stress indicator level of a second user, according to at least one instance of the present disclosure;

FIG. 12A is a data plot of a skin temperature sensor of a wearable device, according to at least one instance of the present disclosure.

FIG. 12 B is a data plot of a context temperature sensor of a wearable device, according to at least one instance of the present disclosure;

FIG. 12C is a data plot of activity sensor of a wearable device, according to at least one instance of the present disclosure;

FIG. 13A is a data plot of ambient light, according to at least one instance of the present disclosure; and

FIG. 13B is a data plot of ambient temperature, according to at least one instance of the present disclosure.

### DETAILED DESCRIPTION

Examples and various features and advantageous details thereof are explained more fully with reference to the exemplary, and therefore non-limiting, examples illustrated in the accompanying drawings and detailed in the following description. Descriptions of known starting materials and processes can be omitted so as not to unnecessarily obscure the disclosure in detail. It should be understood, however, that the detailed description and the specific examples, while indicating the preferred examples, are given by way of illustration only and not by way of limitation. Various substitutions, modifications, additions and/or rearrangements within the spirit and/or scope of the underlying inventive concept will become apparent to those skilled in the art from this disclosure.

### I. Terminology

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “physiological” as used herein (including, but not limited to, terms such as physiological sensors, physiological parameters, physiological changes, and the like) refers to an aspect/characteristic of, or appropriate to, the healthy or normal functioning of a user, specifically with respect to the user's physical or emotional health or wellbeing. Such physiological aspects can be both internal and external to the user.

Additionally, any examples or illustrations given herein are not to be regarded in any way as restrictions on, limits to, or express definitions of, any term or terms with which they are utilized. Instead these examples or illustrations are to be regarded as being described with respect to one particular example and as illustrative only. Those of ordinary skill in the art will appreciate that any term or terms with which these examples or illustrations are utilized encompass other examples as well as implementations and adaptations thereof which can or cannot be given therewith or elsewhere in the specification and all such examples are intended to be included within the scope of that term or terms. Language designating such non-limiting examples and illustrations includes, but is not limited to: “for example,” “for instance,” “e.g.,” “In some examples,” and the like.

Although the terms first, second, etc. can be used herein to describe various elements, components, regions, layers and/or sections, these elements, components, regions, layers and/or sections should not be limited by these terms. These terms are only used to distinguish one element, component, region, layer or section from another. Thus, a first element, component, region, layer or section discussed below could be termed a second element, component, region, layer or section without departing from the teachings of the present inventive concept.

### II. General Architecture

Wearable devices are configured to measure a data point and provide it a user in real-time without providing the user ways to improve the particular data and/or interpret the provided data. The disclosed wearable device offers physiological interventions at predetermined periods of time. The disclosed wearable device monitors whether the user is attempting an intervention and/or if the user is doing the intervention properly.

The systems and methods disclosed herein relate to monitoring and mitigation stress through the use of a wearable device having one or more physiological sensors communicatively coupled therewith.

The wearable device can further be communicatively coupled with one or more context sensors operable to provide data relative to the one or more physiological sensors. The wearable device can detect one or more physiological indicators of stress and suggest a stress intervention to the user to better achieve stress management. The wearable device can monitor compliance with the stress intervention and track whether the stress intervention successfully reduced the one or more physiological indicators of stress.

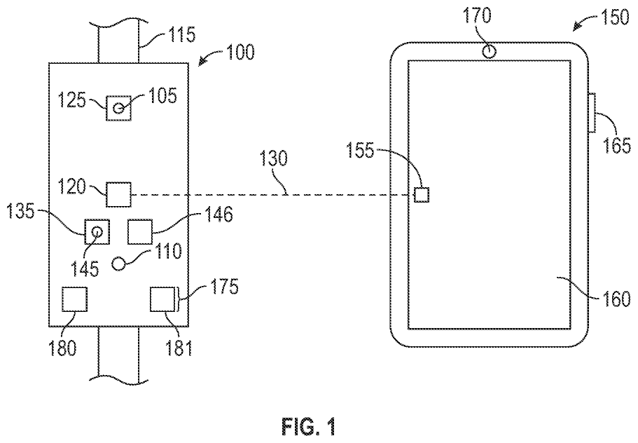

FIG. 1 illustrates a wearable device, according to an instance of the present disclosure. The wearable device 100 can be operably engaged with at least a portion of a user's body. In at least one instance the wearable device 100 can be operably engaged with the user via a band 115. In other instances, the wearable device 100 can be operably engaged with the user via a wearable clothing item (e.g. shirt, pants, shorts, compression sleeve, sock, ring, watch, hat, helmet, patch, etc.)

The portion of the user that the wearable device 100 is operable engaged with can be a plurality of locations including a muscle mass and/or tissue bed, including but not limited to a leg and/or arm of the user. In other instances, the portion of the user that the wearable device 100 is operably engaged with can include, but is not limited to, a finger, a wrist, a head, an ankle, neck, chest, and/or other portion of the user. In at least one instance, the portion of the user that the device is attached can be the wrist for accessibility and ease of use. In another instance, the portion of the user that the device is attached can be the finger for continuous wear. The wearable device 100 can be used with an optional output device 150, such as a smartphone (as shown), a smartwatch, computer, mobile phone, tablet, personal computing device, a generic electronic processing and displaying unit, cloud storage, and/or a remote data repository via a cellular network and/or wireless Internet connection (e.g. Wi-Fi).

The output device 150 can include a display 160 operable to provide a user information and/or data from the one or more physiological sensors (e.g. sensor 125, 135, 175). While the sensors are described herein as being one or more physiological sensors, it should be generally understood that the sensors of the wearable device disclosed herein can monitor any aspect of a user. The sensors, including the one or more physiological sensors, as described herein can include, but are not limited to, an electrodermal (EDA) sensor, a biomechanical sensor, a galvanic skin response (GSR) sensor, a photoplethysmography (PPG) sensor, an electrocardiogram (EKG), an inertial measurement sensor, an accelerometer, a gyroscope, a magnetometer, a global positioning system (GPS), a blood pressure (BP) sensor, a pulse oximetry (SpO2) sensor, a respiratory rate (RR) monitor, a temperature sensor, a humidity sensor, an audio sensor, an air quality sensor, a microphone, an environmental sensor (including but not limited to ambient noise, light, temperature, air quality, humidity, location, ultraviolet (UV) light exposure level, etc.), and/or any other sensor capable of measuring an aspect of a user and/or their environmental surroundings which may affect the user's physical and/or emotional health or wellbeing.

The output device 150 can include an input control device 165 operable to allow a user to change the display 160 and/or the information and/or data displayed thereon. In at least one instance, the input control device 165 can be a button and/or other actuatable element operable to allow an input to be received by the output device 150. In other instances, the input control device 165 can be a touch sensitive input device.

The output device 150 and the wearable device 100 can be communicatively coupled 130 via a transmitter/receiver 120, 155 disposed on the wearable device 100 and the output device 150, respectively. The communicative coupling 130 can be a two-way communication pathway allowing the wearable device 100 to provide information and/or data to the output device 150 and/or the display 160 while similarly allowing the output device 150 to request information and/or data from the wearable device.

One or more context sensors 170 can be disposed on the output device 150 and be operable to provide data regarding a user's ambient environment (e.g. temperature, humidity, light intensity, location, air quality, noise level, ultraviolet light (UV) exposure, etc.).The user's ambient environment can include one or more environmental elements. The one or more context sensors 170 can provide comparative data for the one or more physiological sensors allowing the wearable device 100 to better understand the data measurements from the one or more physiological sensors. While the present disclosure illustrates the one or more context sensors 170 disposed on the output device 150, it is within the scope of this disclosure for the one or more context sensors to be coupled with and/or disposed on the wearable device 100, smart home sensors (e.g. smart thermostat, smart light switch, smart home hub, etc.).

The wearable device 100 can include one or more physiological sensors. The one or more physiological sensors can include, but are not limited to, an electrodemal sensor (EDA), galvanic skin response (GSR) sensor, a photoplethysmography (PPG), an electrocardiogram (EKG), an inertial measurement sensor, an accelerometer, a gyroscope, a blood pressure sensor, a pulse oximetry (SpO2) sensor, a respiratory rate monitor, a temperature sensor, a humidity sensor, an audio sensor, and combinations thereof

The wearable device 100 can include a sensor 125 that is operable to determine a level of a biological indicator within tissue or blood vessels using near-infrared spectroscopy (NIRS). The sensor 125 can include an optical emitter 105 and/or an optical detector 110. The sensor 125 can uses one or more low-power lasers, light emitting diodes (LEDs) and/or quasi-monochromatic light sources and low-noise photodetecting electronics to determine an optical absorption. In another example, the sensor 125 can use a broad-spectrum optical source and a detector sensitive to the spectral components of light, such as a spectrometer, or a charge-coupled device (CCD) or other linear photodetector coupled with near-infrared optical filters.

The wearable device 100 can be configured to include a second sensor 135 operable to measure a photoplethysmography (PPG) of the user. The second sensor 135 can include an optical emitter 145 and/or an optical detector 146. The wearable device 100 can also include a third sensor 175 operable to measure electrocardiography (EKG) and/or derived systolic time intervals (STI) of the user. The third sensor 175 can include a first electrode 180 and/or a second electrode 181. The sensors 125, 135, 175 can each be a physiological sensor of the wearable device, collectively and/or individually. The wearable device 100 can include one or more physiological sensors including, but not limited to, sensors 125, 135, and/or 175, respectively.

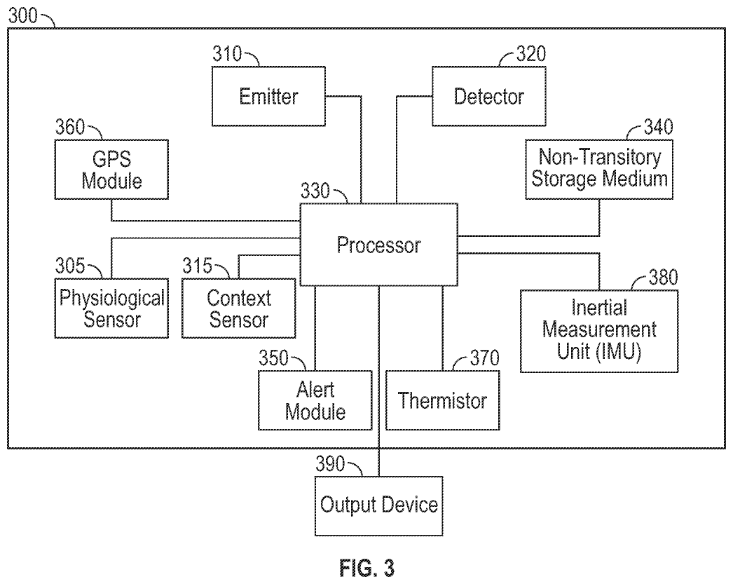

The sensors 125, 135, 175 in the device 100 can measure NIRS parameters, electrocardiography, photoplethysmography, and/or derived systolic time intervals (STI) of the user. The wearable device 100 also includes a processor (shown in FIG. 3) operable to analyze data generated by one or more of the sensors 125, 135, 175 to determine a physiological response and/or physiological change of a user.

In at least one instance, the processor is operable to determine biological indicators, including, but not limited to a relative percentage, a saturation level, an absolute concentration, a rate of change, an index relative to a training threshold, and a threshold. In other instance, the processor is operable to determine perfusion characteristics such as pulsatile rhythm, blood volume, vascular tone, muscle tone, and/or angiogenesis from total hemoglobin and/or water measurements.

The wearable device 100 can include a power supply, such as a battery, to supply power to one or more of the sensors 125, 135, 175 and/or other components in the wearable device 100. In at least one instance, the sensor 125 can be have a skin contact area of approximately 3.5 inches×2 inches. In other instances, the wearable device 100 can be sized to be on the user's wrist so that there is a skin contact area of approximately 1 inch×1 inch. In other instances, the wearable device 100 can be sized to be on the user's finger so that there is a skin contact area of approximately one quarter (¼) inch×one half (½) inch. Additionally, other dimensional skin areas are considered within the scope of this disclosure depending on the number of type of sensors operably implemented with the wearable device 100.

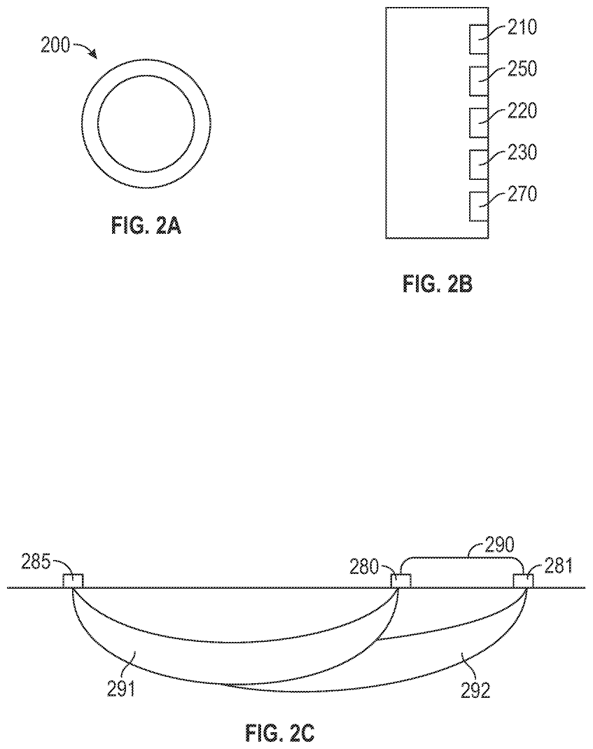

FIG. 2A and 2B illustrates a wearable device having one or more optical physiological sensors, according to at least one instance of the present disclosure. The wearable device 200 can be configured to be worn on a finger of a user. In at least one example, the wearable device 200 can be optimized to a given finger for increased accuracy. The optimization can include physiological sensor selection, arrangement, orientation, and/or shape of the wearable device 200 to ensure proper fitment. In other instances, the wearable device 200 can be optimized based on the size, gender, and/or age of the user. In still other instances, a variety of the above optimizations can be implemented for a given device.

FIG. 2A illustrates a wearable device 200. FIG. 2B illustrates a cross-sectional of the wearable device 200, including emitters 220, 230, 250 and photodetector 210. The wearable device 200 also includes data and/or charging contacts 270. In at least one instance, the data and charging contacts 270 can be operable to electrically detect if the sensor is making contact with the skin of a user. The presence of multiple emitters 220, 230, and/or 250 on the wearable device 200 allows for spatially-resolved data gathering in real-time. The wearable device 200 can be configured to determine the optical absorption of chromophores, such as water, hemoglobin in its multiple forms, including oxyhemoglobin (HbO2), deoxyhemoglobin oxymyoglobin, deoxymyoglobin, cytochrome c, lipids, melanins, lactate, glucose, or metabolites.

FIG. 2C illustrates a spatially-resolved NIRS sensor that can be included on the non-invasive wearable device 200, according to at least one instance of the disclosure. As shown in FIG. 2C, the spatially-resolved NIRS sensor can include light emitters 280 and 281 which emit light that is scattered and partially absorbed by the tissue. Each emitter 280, 281 can be configured to emit a single wavelength of light or a single range of wavelengths. In at least one example, each emitter 280, 281 can be configured to emit at least three wavelengths of light and/or at least three ranges of wavelengths. Each emitter 280, 281 can include one or more light emitting diodes (LEDs). Each emitter 280, 281 can include a low-powered laser, LED, or a quasi-monochromatic light source, and/or any combination thereof. Each emitter 280, 281 can also include a light filter.

A fraction of the light emitted by emitters 280 and 281 can be detected by photodetector 285, as illustrated by the parabolic or “banana shaped” light arcs 291 and 292. Emitters 280, 281, are separated by a known (e.g. predetermined) distance 290 and produce a signal that is later detected at photodetector 285. The detected signal is used to estimate the effective attenuation and absorption coefficients of the underlying tissue. In at least one instance, the known distance 290 is 12 mm. In other instances, the known distance can be selected based on a variety of factors, which can include the wavelength of the light, the tissue involved, and/or the age of the user.

The wearable device 200 disclosed herein can have different numbers of emitters and photodetectors without departing from the principles of the present disclosure. Further, the emitters and photodetectors can be interchanged without departing from the principles of the present disclosure. Additionally, the wavelengths produced by the LEDs can be the same for each emitter or can be different.

In at least one instance, the wearable device 200 can be used for the monitoring of one or more physiological parameters of a user. Use of the wearable device 200 is particularly relevant in endurance type sports, such as running, cycling, multisport competition, rowing, but can also be used in other physical activities. The device 200 can be configured to wirelessly measure real-time physiological parameters continuously throughout the day and/or night. The device 200 can be secured to a selected muscle group, such as the leg muscles of the vastus lateralis or gastrocnemius, or any area of the user where certain physiological parameters are best measured.

FIG. 3 illustrates the components of a wearable device 300 according to at least one instance of the present disclosure. As shown in FIG. 3, the wearable device 300 can include an emitter 310 and detector 320, which can be communicatively coupled to a processor 330. The processor 330 can be communicatively coupled to a non-transitory storage medium 340. The device 300 can be coupled to an output device 390.

The emitter 310 delivers light to the tissue and the detector 320 collects the optically attenuated signal that is back-scattered from the tissue. In at least one instance, the emitter 310 can be configured to emit at least three separate wavelengths of light. In another instance, the emitter 310 can be configured to emit at least three separate bands and/or ranges of wavelengths. In at least one instance, the emitter 310 can include one or more light emitting diodes (LEDs). The emitter 310 can also include a light filter. The emitter 310 can include a low-powered laser, LED, or a quasi-monochromatic light source, or any combination thereof. The emitter can emit light ranging from infrared to ultraviolet light. As indicated above, the present disclosure uses NIRS as a primary example and the other types of light can be implemented in other instances and the description as it relates to NIRS does not limit the present disclosure in any way to prevent the use of the other wavelengths of light.

The data generated by the detector 320 can be processed by the processor 330, such as a computer processor, according to instructions stored in the non-transitory storage medium 340 coupled to the processor. The processed data can be communicated to the output device 390 for storage or display to a user. The displayed processed data can be manipulated by the user using control buttons or touch screen controls on the output device 390.

The wearable device 300 can include an alert module 350 operable to generate an alert including, but not limited to a suggested response to a physiological change. The processor 330 can send the alert to the output device 390 and/or the alert module 350 can send the alert directly to the output device 390. In at least one instance, the processor 330 can be operably arranged to send an alert to the output device 390 without the wearable device 300 including an alert module 350.

The alert can provide notice to a user, via a speaker or display on the output device 390, of a change in one or more physiological conditions or other parameter being monitored by the wearable device 300, or the alert can be used to provide an updated stress level to a user. In at least one instance, the alert can be manifested as an auditory signal, a visual signal, a vibratory signal, or combinations thereof. In at least one instance, an alert can be sent by the processor 330 when a predetermined physiological change occurs.

In at least one instance, the wearable device 300 can include a Global Positioning System (GPS) module 360 configured to determine geographic position and tagging the biological and/or physiological data with location-specific information. The wearable device 300 can also include a thermistor 370 and an IMU 380. The IMU 380 can be used to measure, for example, a gait performance of a walker and/or runner and/or a pedal kinematics of a cyclist, as well as one or more physiological parameters of a user. The thermistor 370 and IMU 380 can also serve as independent sensors configured to independently measure parameters of physiological threshold. The thermistor 370 and IMU 380 can also be used in further algorithms to process or filter the optical signal.

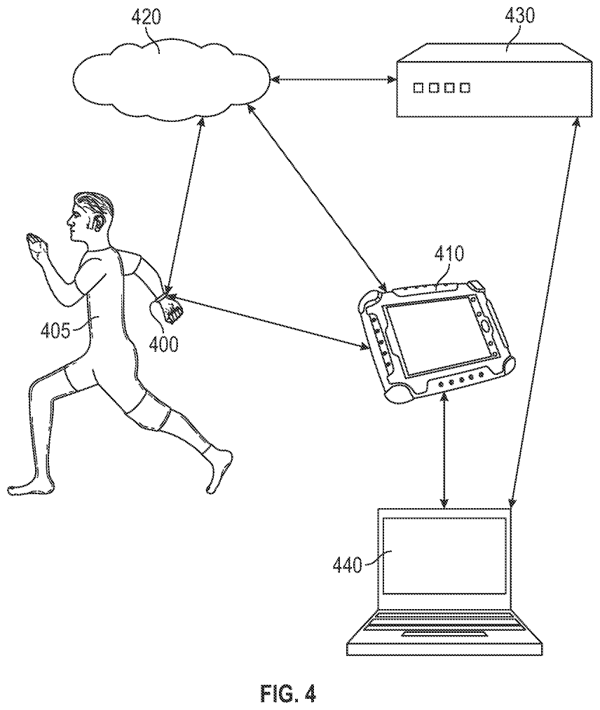

FIG. 4 illustrates an environment within which the wearable device can be implemented, according to at least one instance of the present disclosure. As shown in FIG. 4, the wearable device 400 is worn by a user to determine one or more biological and/or physiological indicator levels. The wearable device 400 is depicted as being worn on the wrist of a user 405; however, the wearable device 400 can be worn on any portion of the user suitable for monitoring biological and/or physiological indicator levels. The wearable device 400 can be used with an output device 410, such as a smartphone (as shown), a smart watch, computer, mobile phone, tablet, a generic electronic processing and/or displaying unit, cloud storage, and/or a remote data repository via a cellular network or wireless Internet connection.

As shown in FIG. 4, the wearable device 400 can communicatively couple with a output device 410 so that data collected by the wearable device 400 can be displayed and/or transferred to the output device 410 for communication of real-time biological and/or physiological data to the user 405. In at least one instance, an alert can be communicated from the device 400 to the output device 410 so that the user 405 can be notified of a biological and/or physiological event. Communication between the wearable device 400 and the output device 410 can be via a wireless technology, such as BLUETOOTH®, infrared technology, or radio technology, and/or can be through a wire. Transfer of data between the wearable device 400 and/or the output device 410 can also be via removable storage media, such as a secure digital (SD) card. In at least one instance, a generic display unit can be substituted for the output device 410.

The wearable device 400 can communicatively couple with a personal computing device 440 and/or other device configured to store or display user-specific biological and/or physiological indicator data. The personal computing device 440 can include a desktop computer, laptop computer, tablet, smartphone, smart watch, or other similar device. Communication between the wearable device 400 and the personal computing device 440 can be via a wireless technology, such as BLUETOOTH®, infrared technology, or radio technology. In other instances, the communication between the wearable device 400 and the personal computing device 440 can be through a wire and/or other physical connection. Transfer of data between the wearable device 400 and the personal computing device 440 can also be via removable storage media, such as an SD card.

The output device 410 can communicate with a server 430 via a network 420, allowing transfer of user-specific biological and/or physiological data to the server 430. The output device 410 can also communicate user-specific biological and/or physiological data and/or physiological data to cloud-based computer services or cloud-based data clusters via the network 420. The output device 410 can also synchronize user-specific biological and/or physiological data with a personal computing device 440 or other device configured to store or display user-specific biological and/or physiological data. The output device 410 can also synchronize user-specific biological and/or physiological data with a personal computing device 440 or other device configured to both store and display user-specific biological and/or physiological data. Alternatively, the personal computing device 440 can receive data from a server 430 and/or cloud-based computing service via the network 420.

The personal computing device 440 can communicate with a server 430 via a network 420, allowing the transfer of user-specific biological and/or physiological data to the server 430. The personal computing device 440 can also communicate user-specific biological and/or physiological data to cloud-based computer services and/or cloud-based data clusters via the network 420. The personal computing device 440 can also synchronize user-specific biological and/or physiological data with the output device 410 and/or other device configured to store or display user-specific biological and/or physiological data.

The wearable device 400 can also directly communicate data via the network 420 to a server 430 or cloud-based computing and data storage service. In at least one instance, the wearable device 400 can include a GPS module configured to communicate with GPS satellites (not shown) to obtain geographic position information.

The wearable device 400 can be used by itself and/or in combination with other electronic devices and/or context sensors. The context sensors can include, but are not limited to, sensors coupled with electronic devices other than the wearable device 400 including smart devices used both inside and outside of a home. In at least one instance, the wearable device 400 can be used in combination with heart rate (HR) biosensor devices, foot pod biosensor devices, and/or power meter biosensor devices. In at least one instance, the wearable device 400 can also be used in combination with ANT+™ wireless technology and devices that use ANT+™ wireless technology. The wearable device 400 can be used to aggregate data collected by other biosensors including data collected by devices that use ANT+™ technologies. Aggregation of the biosensor data can be via a wireless technology, such as BLUETOOTH®, infrared technology, or radio technology, or can be through a wire.

The biosensor data aggregated by the wearable device 400 can be communicated via a network 420 to a server 430 or to cloud-based computer services or cloud-based data clusters. The aggregated biosensor data can also be communicated from the wearable device 400 to the output device 410 or personal computing device 440.

In at least one instance, the wearable device 400 can employ machine learning algorithms by comparing data collected in real-time with data for the same user previously stored on a server 430, output device 410, and/or in a cloud-based storage service. In other instances, the wearable device 400 can compare data collected in real-time with data for other users stored on the server 430 and/or in cloud based storage service. The machine learning algorithm can also be performed on or by any one of the output device 410, cloud-based computer service, server 430, and/or personal computing device 440, and/or any combination thereof.

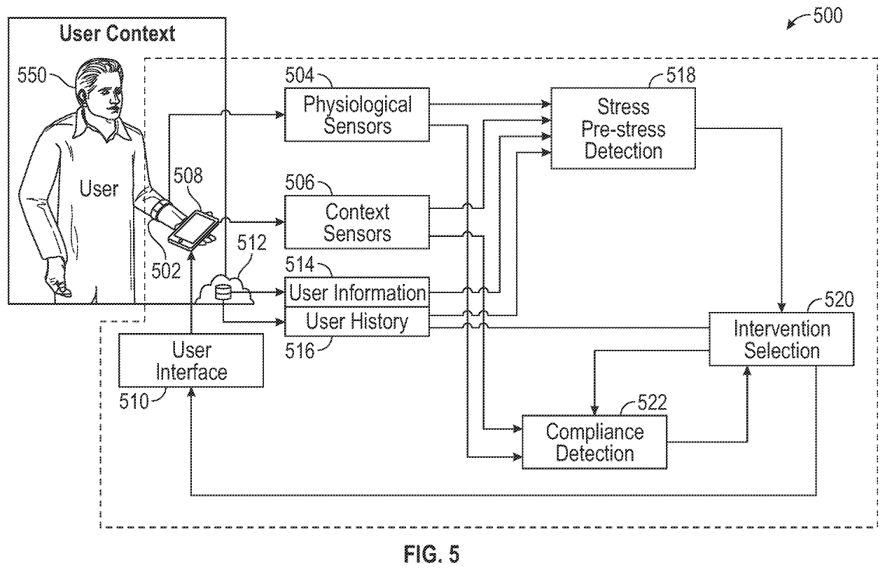

FIG. 5 illustrates an example wearable device system operable to detect and manage a stress level of a user. The wearable device 502 can include one or more physiological sensors 504 operable engaged with the user and operably coupled with a wearable device system 500. The one or more physiological sensors 504 can include, but are not limited to, an electrodermal sensor (EDA) sensor, a photoplethysmography (PPG) sensor, an electrocardiogram (EKG) sensor, an inertial measurement (IMU) sensor, an accelerometer, a gyroscope, a blood pressure sensor, a pulse oximetry (SpO2) sensor, a respiratory rate monitor, a temperature sensor, a humidity sensor, an audio sensor, and/or combinations thereof. The one or more physiological sensor 504 can be an optical sensor including active and/or passive camera systems operable to quantify blood pulse volume, blood pressure, heart rate, heart rate variability, and/or optically opaque compounds (e.g. hemoglobin, etc.).

The one or more physiological sensors 504 can include thermal systems operable to measure temperature via infrared systems and/or thermocouples. Sweat quantification systems can be galvanic skin response and/or EDA. Pressure system can be implemented to monitor blood pressure, and motion system can be implemented to monitor user 550 movement including, but not limited to, inertial measurement unit (IMU), accelerometer, gyroscope, magnetometer, and/or GPS.

The wearable device 502 can be a watch, wristband, ring, necklace, clothing (e.g. shirt, sock, underwear, bra, compression sleeve, etc.), adhesive patch, continuous glucose monitors (CGM), other medical equipment, and/or combinations thereof. Additionally, the wearable device 502 can be implemented to include one or more of the features described above with respect to wearable devices illustrated in FIGS. 1-4.

The wearable device system 500 can be communicatively coupled with one or more context sensors 506 operably coupled with the wearable device 502. The one or more context sensors 506 can provide the wearable device system 500 with information about a user's ambient environment and/or location. The one or more context sensors 506 can provide ambient temperature, ambient light intensity, ambient humidity, and/or location. The one or more context sensors 506 can be disposed on the wearable device 502 and/or communicatively coupled with the wearable device 502. In at least one instance, the one or more context sensors 506 can include a smartphone operable to provide location information of the user. In other instances, the one or more context sensors 506 can include a smart thermostat operable provide ambient temperature information (e.g. room temperature), a smart light switch operable to provide ambient light intensity information, a smart hub operable to provide location information within a home, bathroom fixtures (e.g. scale, mirror, toilet with sensors, etc.), smart microphones, smart refrigerators, vehicles, and/or combinations thereof.

The wearable device system 500 can utilized the one or more context sensors 506 to appropriate characterize and/or provide prospective to the physiological data of the one or more physiological sensors 504.

The wearable device system 500 can further include a display 508 operable to engage with the user 550. In at least one instance, the display 508 can be a user's smartphone and can be independent of but communicatively coupled with the wearable device 502. The display 508 can provide a user interface 510 through which a user 550 interacts with the wearable device system 500.

A server 512 can be communicatively coupled with the wearable device 502 and can be operable to store user information 514 and/or user history 516. The user information 514 and/or user history 516 can be include input personal information about the user (e.g. height, weight, age, gender, medical history, etc.) and/or stored measurements obtained from the one or more physiological sensors 504 and/or the one or more context sensors 506.

The server 512 can be a conventional physical server and/or a cloud-based server storage solution.

The wearable device 502 can determine a stress or pre-stress detection 518 via measurements from the one or more physiological sensors 504 and/or the one or more context sensors 506. The stress or pre-stress detection 518 can be indicated by changes in one or more physiological response by the user 550 (e.g. increased perspiration) while accounting for the user's environment through the one or more context sensors 506. The stress or pre-stress detection 518 can have a predetermined threshold for stress indication in view of the user information 514 and/or user history 516 and/or collective user data obtained through a cloud storage solution.

Stress can be measured and/or determined from the one or more physiological sensors by determining a physiological change and/or combination of physiological changes experienced by a user. Examples of indications of stress include, but are not limited to, increased heart rate (not caused by physical activity), increases in breathing rate, decrease in skin temperature due to sweating and/or peripheral vasoconstriction without a decrease in ambient temperature (via the one or more context sensors 506), increases in glucose without recent food ingestion, increases in skin conductivity and rate of sweat glad activation without physical activity, decrease in peripheral perfusion, decrease in heart rate variability (e.g. a more regular heart beat), increase in blood pressure, movement deviation away from a normal patter (e.g. pacing), changes in vocalizations (e.g. shouting, yelling, and/or tone), and/or combinations thereof.

Upon detection of a stress or pre-stress above the predetermined threshold, the wearable device 502 can offer a stress intervention selection 520. In at least one instance, the stress intervention selection 520 can a few options operable to reduce a stress index of the user as measured by the one or more physiological sensors and allow the user to select a desired stress intervention 520. In other instances, the stress intervention selection 520 can be a single option operable to reduce a stress index.

As the user participates in the stress intervention selection 520, the wearable device 502 can have compliance detection 522 to determine if the user is participating in the stress intervention selection 520 appropriately. In at least one instance, the stress intervention selection 520 can be a box breathing exercise and the compliance detection 522 can monitor the user's 550 breathing pattern and/or respiration rate to determine if the user is following the box breathing exercise. In other instances, the stress intervention selection 520 can be talking a walk outdoors and the one or more context sensors 506 and/or the one or more physiological sensors 504 can be monitored to determine if the user's 550 location, ambient temperature, gait, heart rate, etc. changed, thereby indicating the user 550 is taking a walk. If the compliance detection 522 determine the user 550 is not complying with the stress intervention selection 520, the intervention selection 520 can be continued and/or repeated until the compliance detection 522 determines the user 550 has succeeded in completing the stress intervention selection 520.

The wearable device 502 monitors the stress or pre-stress detection 518 before, during, and/or after the stress intervention selection 520, and can determines if the stress or pre-stress detection dropped below the predetermined threshold following the stress intervention selection 520. If the stress index did not drop below the predetermined threshold, the user 550 can be recommended to complete another stress intervention selection 520. In at least one instance, the subsequent stress intervention selection 520 can be a new exercise or activity. The wearable device system 500 can monitor, track, and learn which stress intervention selections 520 work for a particular user 550 and recommend them more regularly than other stress intervention selections 520. In at least one instance, the wearable device system 500 can be operable to determine different types of stress indicated by the one or more physiological sensors 502, and recommend varying stress intervention selections 520 based on the type of stress detected. The types of stress can be determined based on the user physiological response as measured by the one or more physiological sensors 504 (e.g. heart rate, temperature, perspiration, etc.).

In some instances, the user interface 510 can be operable to guide the user 550 through the stress intervention selection 520 by illustrating a video, diagram, and/or other graphic. The user interface 510 can provide the user 550 instructions and/or demonstration for a stress intervention selection 520. In at least one instance, the user interface 510 can provide a box breathing video demonstrating how the technique is performed, while also indicating when a user 550 inhale and exhale, as appropriate. The user interface 510 can thus assist in ensuring compliance with the intervention selection.

The stress intervention selection 520 can alternatively be meditation, walk, exercise, movement, music, videos, journal exercise, psychotherapy (including cognitive behavioral therapy (CBT)), acts of kindness, social connections and/or interactions.

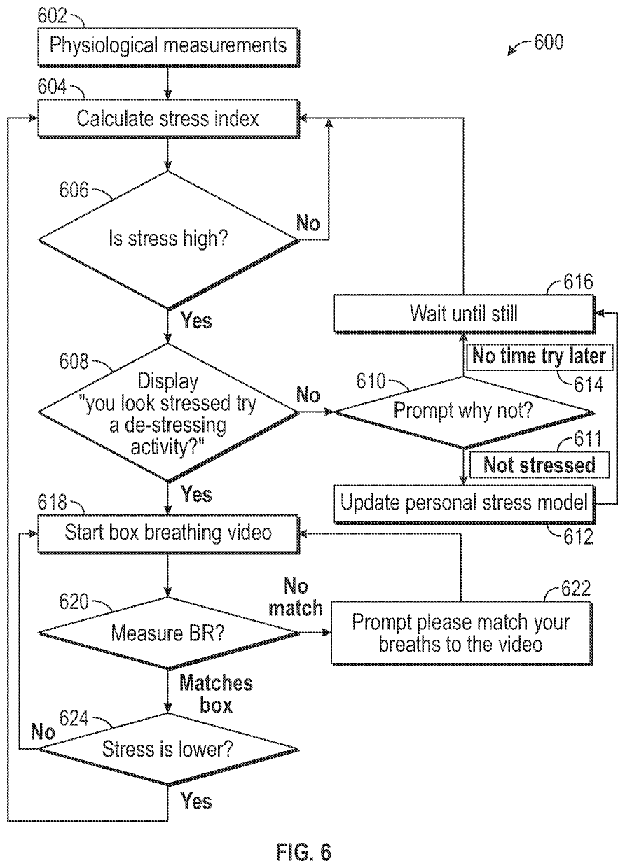

FIG. 6 illustrates a flowchart of a stress management system operable to be implemented with a wearable device, according to at least one instance of the present disclosure. A wearable device having one or more physiological sensors operably coupled therewith can obtain one or more physiological measurements 602 from the one or more physiological sensors, for example those described above with respect to FIGS. 1-5.

The one or more physiological measurements 602 can be utilized to calculate a stress index 604 of a user. The stress index 604 can gauge whether the user is experiencing stress based on changes to one or more of the physiological measurements. The wearable device and/or wearable device system can determine if the stress index 604 exceeds a predetermined threshold 606. The predetermined stress threshold can be determined by baseline data obtained from a plurality of user's via a cloud computing network, or from baseline data obtained from the user over a predetermined period of time, thereby training the wearable device. If the stress index does not exceed the predetermined threshold, the wearable device can wait a predetermined period of time prior to recalculating a stress index 604 based on the one or more physiological measurements 602.

If the stress index 604 exceeds the predetermined threshold 606, thereby indicating an elevated stress level, the wearable device can display a notification 608 regarding the elevated stress level. The notification can request a user participate in a de-stressing activity aimed at reducing the stress index 604 and monitored via the one or more physiological measurements 602.

The user can decline 610 to participate in the de-stressing activity, in which the wearable device can request feedback relating to the reason for declining the de-stressing activity. In one instance, the user can indicate they are not stressed and the wearable device can update the personal stress indication model 612, further training stress index 604 of the wearable device for the user. In other instances, the user can indicate no time 614 to participate in the de-stressing exercise, and the wearable device can wait a predetermined period of time 616 prior to repeating the process. The wearable device can wait the predetermined period of time 616 prior to requesting the user participate in the de-stressing exercise, and/or can wait the predetermined period of time 616 before re-calculating the stress index 604 to determine stress level.

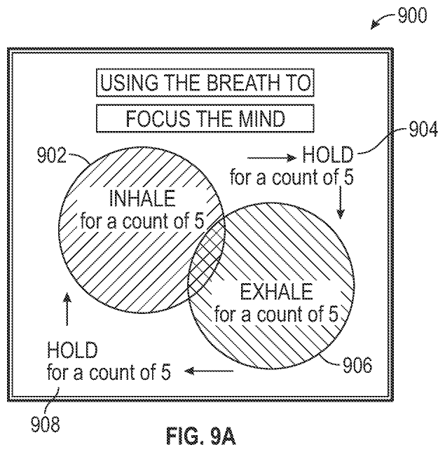

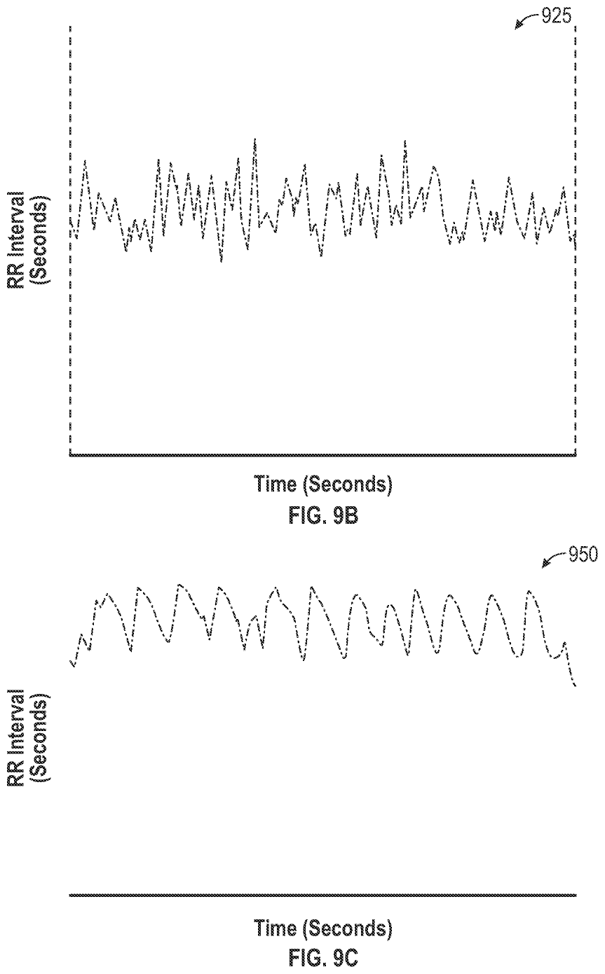

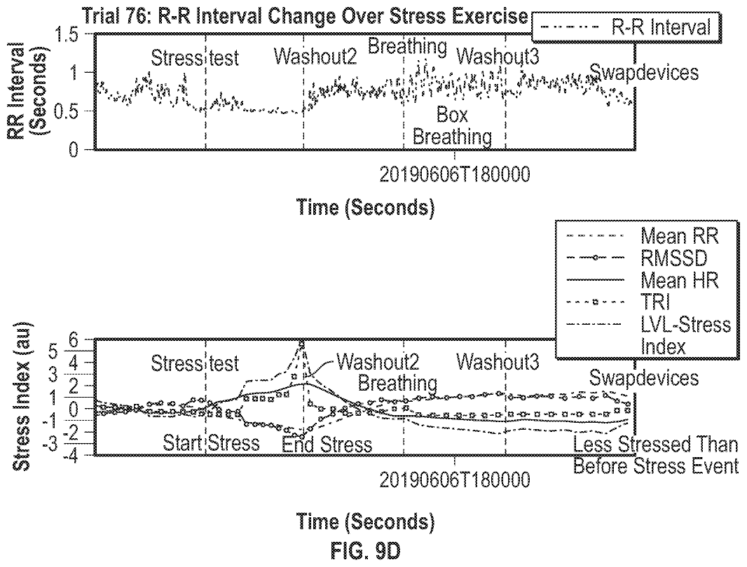

If the user elects to participate in a de-stressing exercise, the wearable device can recommend one or more de-stressing activities and/or exercises 618. The de-stressing activities and/or exercises 618 can be a breathing exercise (e.g. box breathing), taking a walk outdoors, listening to music, meditating, and/or the like. As can be appreciated in FIG. 6, the wearable device de-stressing recommendation can be to play an instructional box breathing video. As the user participates in the de-stressing activity (e.g. box breathing shown with respect to FIG. 9A), the wearable device can monitor compliance 620 with the instructions, recommendations, and/or process. In at least one instance, the wearable device monitors compliance 620 with the box breathing exercise by monitoring a user's respiration rate by the one or more physiological sensors.

If the user fails to comply with the de-stressing activity instructions, the wearable device can prompt the user to follow the instructions 622 (e.g. match your breathes to the box breathing video). If the user complies with the de-stressing activity instructions, the wearable device can determine if the stress index was lowered 624. If the stress index was lowered, the wearable device can revert to calculating stress index 604 to determine if and/or when a user becomes stressed in the future. If the stress index was not lowered 626 following compliance with the de-stressing activity instructions, the wearable device can start another de-stressing activity. In at least one instance, the wearable device can recommend the user repeat the previous de-stressing activity. In other instances, the wearable device can recommend a different de-stressing activity.

The wearable device can train and/or learn, via machine learning algorithms and/or the one or more processors, which de-stressing activities are successful in de-stressing the user, and can recommend the successful de-stressing activities more regularly.

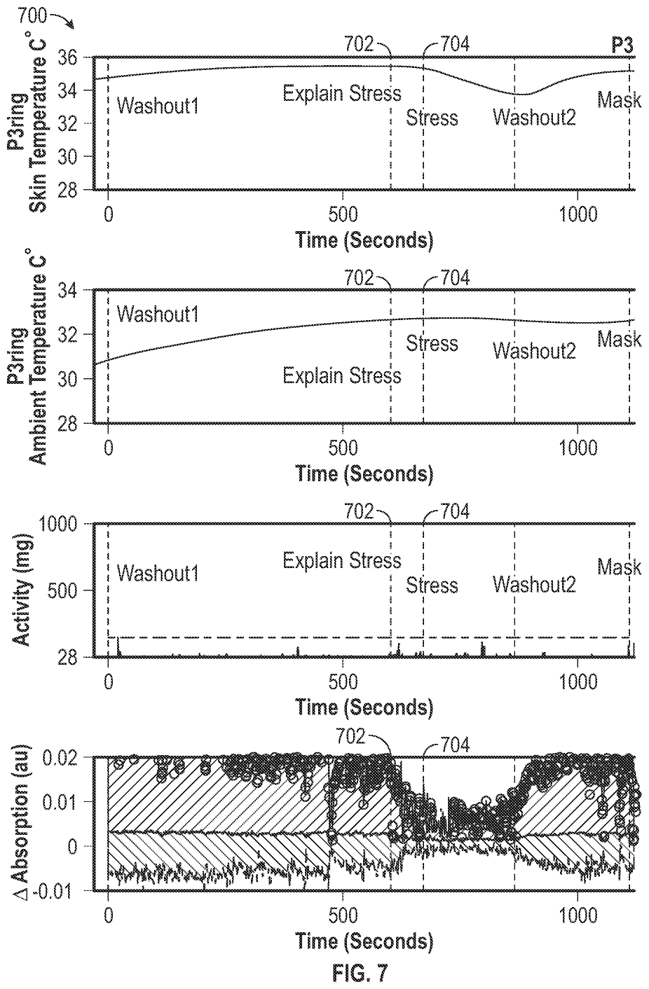

FIG. 7 illustrates a response to stress over time as measured by the one or more physiological sensors, according to at least one instance of the present disclosure. The data plot 700 provides a skin temperature, ambient temperature, activity, and pulse perfusion measurements over time as measured by the one or more physiological sensors of the wearable device. The pulse perfusion can be a measure of how much the absorbance of the blood changes with each beat of the heart. Higher levels can indicate more blood is seen by the sensor and thus more peripheral perfusion. As can be appreciated in FIG. 7, the second dotted line 702 labelled “Explaining Stress” shows a reduction of the peripheral perfusion from an explanation of an upcoming stressful encounter. At the third dotted line 704 labelled “Stress”, the peripheral perfusion continues to decrease along with the skin temperature, despite the ambient temperature staying relatively stable. The decline in skin temperature can indicate sweating and/or perspiration, which can provide an indicate of stress in the user.

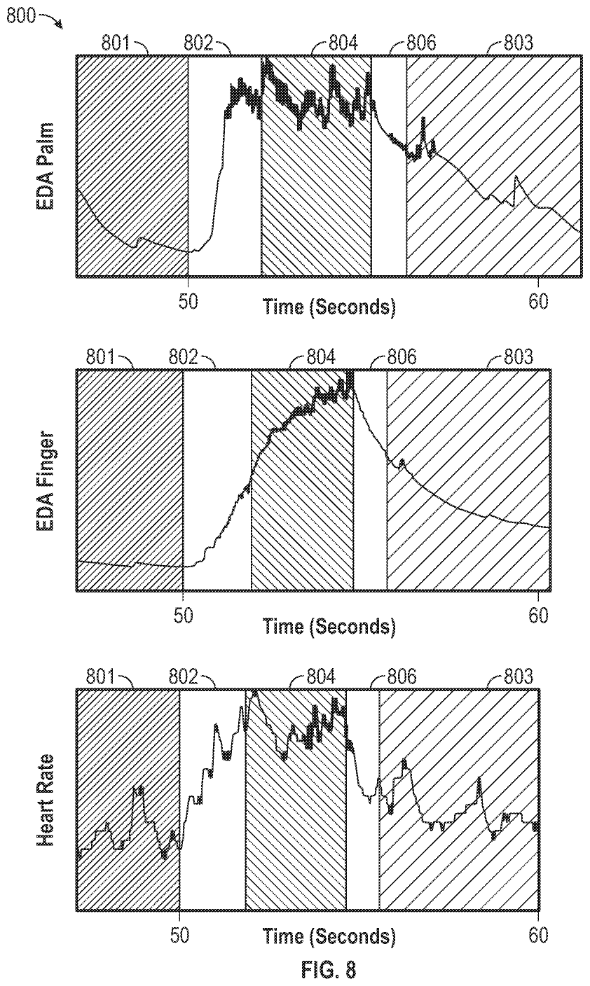

FIG. 8 illustrates a response to stress over time as measured by the one or more physiological sensors regarding EDA palm, EDA finger, and heart rate, according to at least one instance of the present disclosure. The wearable device having one or more physiological sensors including a palm EDA, finger EDA, and heart rate sensor can be operably tracked to determined stress in a user and the response to stress. The monitoring of pre-stress, stress, and post-stress can assist the wearable device in understanding a particular user's indicators of stress and/or their physiological response to de-stressing. The beginning and ending regions 801, 803 show “wash out” periods before and after the stressful period. The period immediately proceeding the stress 802, stressful period 804 is where the upcoming stressful encounter is explained. In response to stress 804 and pre-stress 802, we see an increase in skin conductance level (SCL) as well as an increase in heart rate. The period post-stress 806 illustrates the skin conductance and heart rate return to a more normal level prior to a full return to normalcy in the ending region 803.

FIG. 9A illustrates a box breathing stress intervention activity, according to at least one instance of the present disclosure. A box breathing exercise 900 can be utilized to as a stress intervention activity following detection by the wearable device that a user is experiencing stress. The box breathing exercise 900 include an inhale 902 portion followed by an exhale portion 904. The inhale portion 902 can instruct the under to inhale for a predetermined number of seconds (e.g. to a count of 5) and then to a hold portion 904 in which the inhaled breath can be maintained for a similar predetermined number of seconds. The user can then be instructed to proceed to the exhale portion 906 in which the user exhales for a predetermined number of seconds (e.g. to a count of 5) and then to a hold portion 908 in which the inhaled breath can be maintained for a similar predetermined number of seconds.

While box breathing is illustrated as a specific example of a breathing exercise, it is within the scope of this disclosure to implement any number of breathing exercises including, but not limited to, pursed lip breathing, belly breathing, breath focus, lion's breath, alternate nostril breathing, equal breathing, resonant breathing, sitali breath, deep breathing, and/or humming bee breath. The wearable device can be operable provide instruction on the breathing exercise and/or monitor the user's compliance with the breathing exercise through respiration monitoring via the one or more physiological sensors.

The box breathing can follow this pattern for one or more minutes, allowing the user to develop a specific respiration pattern. The wearable device can monitor the user's respiration in view of the box breathing exercise 900 instructions to monitor for compliance with the instructions and proper execution of the box breathing exercise 900. As can be illustrated by FIG. 9B, the wearable device can determine non-compliant breathing pattern 925. As can further be appreciated in FIG. 9C, the wearable device can similarly determine a compliant breathing pattern 950. The non-compliant breathing pattern 925 shows an irregular respiration rate (RR), which is inconsistent with the box breathing exercise 900. The compliant breathing pattern 950 illustrates a deep, regular respiration rate indicative of the box breathing exercise 900, thereby indicating the user was properly executing the exercise and following the instructions.

FIG. 9D illustrates a respiration rate compared with a heart rate and stress index, according to at least one instance of the present disclosure. As can be appreciated in FIG. 9D, the physiological measurement displayed as the respiration rate interval can be collected from the wearable device using one or more physiological sensors, such as an EKG sensor and/or other ppg based techniques. The calculated metrics of the stress index can be displayed in view of the one or more physiological sensors and the calculated HRV metrics that are derived from the raw respiration rate intervals and are combined to form the stress index. FIG. 9D details the pre-stress, stress, stress intervention activity, and post-stress response as monitored by the wearable device

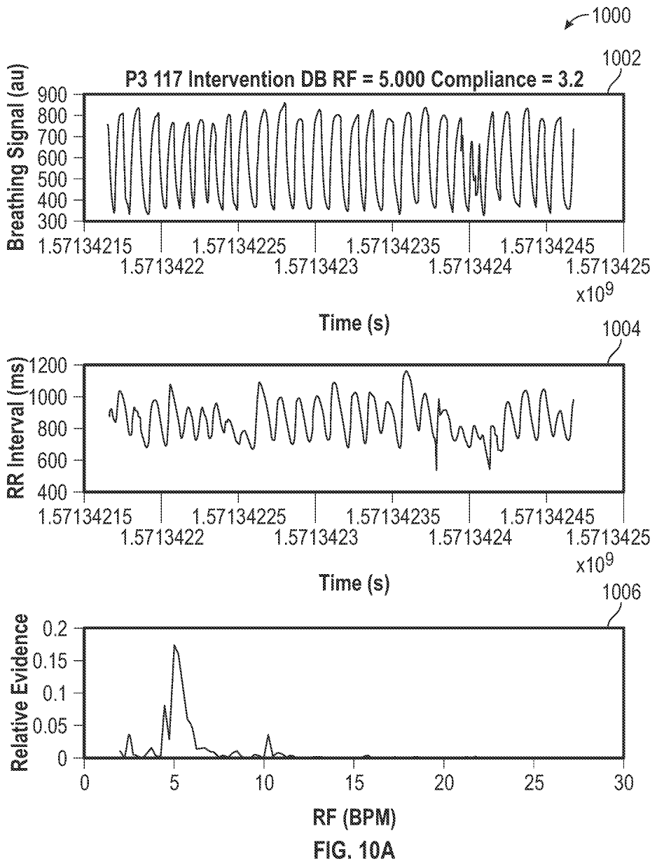

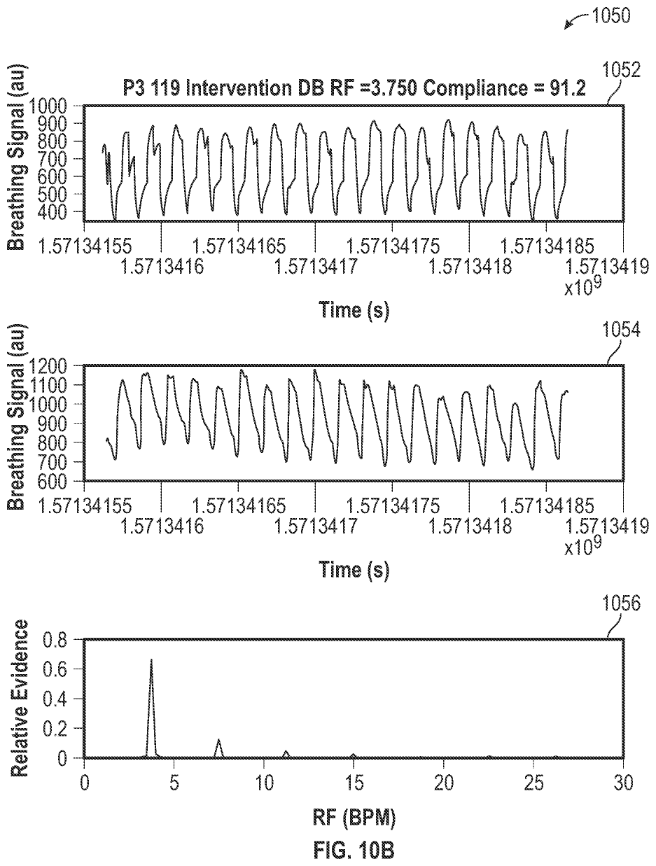

FIG. 10A illustrates a data plot of a deep self-paced breathing signal 1000, frequency modulation of heart rate caused by lung inflation, and a frequency content of the frequency modulation, according to at least one instance of the present disclosure. FIG. 10B illustrates a data plot of a box breathing signal 1050, frequency modulation of heart rate caused by lung inflation, and a frequency content of the frequency modulation, according to at least one instance of the present disclosure. As can be appreciated in FIG. 10A and FIG. 10B each plot shows three rows, a direct measurement of breathing using a sensor in front of both the nose and mouth to detect inhalations and exhalations 1002, 1052, a Frequency Modulation of the heart rate caused by lung inflation changing the normal rhythm of the heart 1004, 1054, and the frequency content of the FM modulation 1006, 1056. The frequency content of the FM modulation matches the breathing above. Patterns in the frequency content of the FM modulation, as well as correlations between the expected breathing pattern and the observed breathing pattern allow the measurement of breathing compliance.

FIG. 10A represents a self-paced deep breaths 1000 that match the gross breathing rate of the prescribed “box breathing” exercise, as illustrated in FIG. 9A. In FIG. 10A, the user is inhaling and/or exhaling at the appropriate times, but otherwise not following the instructional exercise. If the wearable device and/or related system simply checked that the breathing rate or the number of breaths was “good” this breathing pattern would be accepted as maintaining compliance. However, the user did not actually follow the exercise as prescribed, and thus the stress reduction benefits could be impacted. FIG. 10B illustrates the proper box breathing pattern with the very distinct frequency pattern 1052, 1054 caused by this exercise.

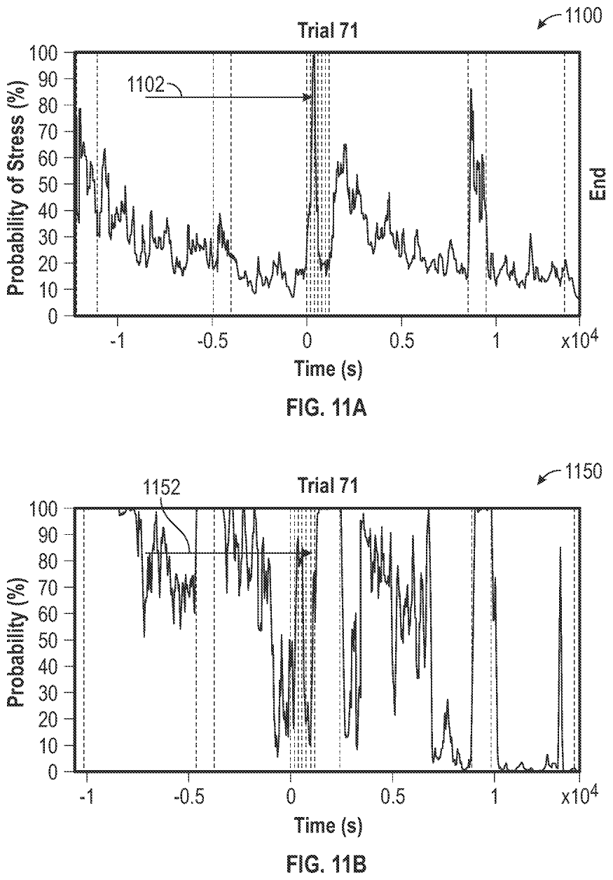

FIG. 11A illustrates a first user stress level over a predetermined time. FIG. 11B illustrates a second user stress level over a predetermined time. FIG. 11A and FIG. 11B illustrate a similar predetermined period of time (e.g. 8 hour work day) for two separate user 1100, 1150. Each user participated in the same activities including a “stressful” event denoted by the arrow 1102, 1152. FIG. 11A illustrates a user having a good stress reactivity and can rapidly recover from stressful events. The need for intervention is usually low and would only be valuable when stress levels are of high intensity and/or long duration. FIG. 11B illustrates the user represents poor stress reactivity. The user responds to stressful events more quickly and those stressful events last longer. Interventions are likely valuable at lower levels of stress and as soon as stress is detected as the user will not recover from stress on their own.

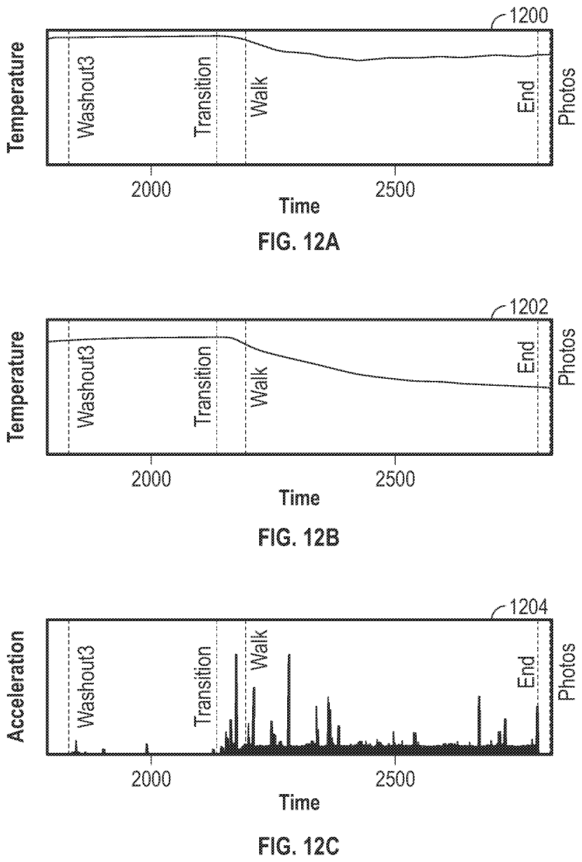

FIG. 12A illustrates a skin temperature measurement, according to at least one instance of the present disclosure. FIG. 12B illustrates an ambient temperature, according to at least one instance of the present disclosure. FIG. 12C illustrates an activity plot, according to at least one instance of the present disclosure. A skin temperature plot 1202 can illustrate the user taking a walk outside during a winter season. As can be appreciated in the skin temperature plot 1202, the skin temperature drops during the activity. The skin temperature plot 1202 can be generated by the one or more physiological sensors associated with the wearable device. An ambient temperature plot 1204 illustrates an environmental temperature the user is experiencing during the walk (or other activity) due to the winter season. The ambient temperature plot 1204 can be generated by the one or more context sensors associated with the wearable device. An activity plot 1206 illustrates that the user increased their activity as the ambient skin temperature plot 1202 and the ambient temperature plot 1204 decrease, which leads the wearable device and/or related system to determine the user followed an activity instruction to take a walk outside during the winter season.

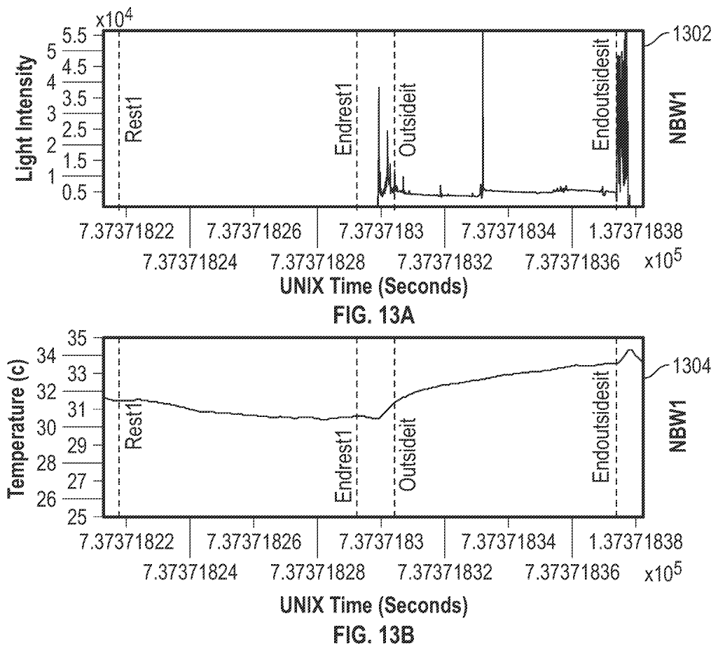

FIG. 13A illustrates an ambient light measurement, according to at least one instance of the present disclosure. FIG. 13B illustrates an ambient temperature measurement, according to at least one instance of the present disclosure. As can be appreciated in FIG. 13A and FIG. 13B, the ambient light measurement in conjunction with the ambient temperature measurement can be utilized to detect a “going outside” activity instruction during a warmer season. The user has gone outside, thus demonstrating an increase in both ambient light and ambient temperature, which leads the wearable device and/or related system to determine the user followed an activity instruction to go outside during the warmer season

While preferred examples of the present inventive concept have been shown and described herein, it will be obvious to those skilled in the art that such examples are provided by way of example only. Numerous variations, changes, and substitutions will now occur to those skilled in the art without departing from the disclosure. It should be understood that various alternatives to the examples of the disclosure described herein can be employed in practicing the disclosure. It is intended that the following claims define the scope of the disclosure and that methods and structures within the scope of these claims and their equivalents be covered thereby.

### Statement Bank

Statement 1: A stress management apparatus comprising: a wearable device having one or more physiological sensors operable to be engaged with a body of a user; one or more processors communicatively coupled with the wearable device, the one or more processors having a memory storing instructions when executed operable to: detect one or more physiological indicators of stress; suggest a stress intervention to the user; monitor a compliance with the stress intervention; and track a reduction of the one or more physiological indicators of stress.

Statement 2: The stress management apparatus of Statement, wherein the one or more physiological sensors are an electrodemal sensor (EDA), galvanic skin response (GSR) sensor, a photoplethysmography (PPG), an electrocardiogram (EKG), an inertial measurement sensor, an accelerometer, a gyroscope, a blood pressure sensor, a pulse oximetry (SpO₂) sensor, a respiratory rate monitor, a temperature sensor, a humidity sensor, an audio sensor, and combinations thereof

Statement 3: The stress management apparatus of Statement 1 or Statement 2, wherein the one or more physiological indicators of stress are a change of one or more of skin temperature, heart rate, heart rate variability, blood pulse volume, skin conductance, skin impedance, blood pressure, breathing rate, blood oxygenation, and/or perspiration.

Statement 4: The stress management apparatus of any one of Statements 1-3, wherein a stress intervention is a breathing exercise having a predetermined sequence of inhale and/or exhale patterns.

Statement 5: The stress management apparatus of any one of Statements 1-4, wherein monitoring a compliance with the stress intervention monitors an inhale and/or exhale pattern of the user compared with the inhale and/or exhale pattern associated with the breathing exercise.

Statement 6: The stress management apparatus of any one of Statements 1-5, further comprising repeat the stress intervention if the compliance with the stress intervention is below a predetermined threshold.

Statement 7: The stress management apparatus of any one of Statements 1-6, further comprising one or more context sensors, wherein the one or more context sensors are operable to detect one or more environmental elements of the user.

Statement 8: The stress management apparatus of any one of Statements 1-7, wherein the one or more environmental elements are ambient temperature, humidity, air quality, noise level, ultraviolet (UV) level, and/or location.

Statement 9: The stress management apparatus of any one of Statements 1-8, wherein the one or more physiological indicators of stress are determined relative to the one or more environmental elements.

Statement 10: The stress management apparatus of any one of Statements 1-9, further comprising determine if the one or more physiological indicators of stress has changed.

Statement 11: The stress management apparatus of any one of Statements 1-10, further comprising establish a baseline for the one or more physiological indicators of stress in an unstressed state for the user.

Statement 12: The stress management apparatus of any one of Statements 1-11, wherein the baseline is adjusted through continuous monitoring of the one or more physiological sensors.

Statement 13: A method of stress management, the method comprising: detecting, via one or more physiological sensors, one or more physiological indicators of stress; suggesting a stress intervention to a user; monitor, via the one or more physiological sensors, compliance with the stress intervention; and tracking, via the one or more physiological sensors, a reduction of the one or more physiological indicators of stress.

Statement 14: The method of stress management of Statement 13, further comprising establishing, via the one or more physiological sensors, a baseline for the one or more physiological indicators of stress in an unstressed state for the user.

Statement 15: The method of stress management of Statement 13 or Statement 14, further comprising adjusting the baseline of the one or more physiological indicators of stress continuously.

Statement 16: The method of stress management of any one of Statements 13-15, further comprising repeating the stress intervention if the compliance with the stress intervention is below a predetermined threshold.

Statement 17: The method of stress management of any one of Statements 13-16, wherein the one or more physiological indicators of stress are determined relative to one or more environmental elements operably detected by one or more context sensors.

Statement 18: The method of stress management of any one of Statements 13-17, wherein the one or more environmental elements are ambient temperature, humidity, air quality, noise level, ultraviolet (UV) level, and/or location.

Statement 19: The method of stress management of any one of Statements 13-18, wherein a stress intervention is a breathing exercise having a predetermined sequence of inhale and/or exhale patterns.

Statement 20: The method of stress management of any one of Statements 13-19, wherein monitoring compliance with the stress intervention monitors an inhale and/or exhale pattern of the user compared with the inhale and/or exhale pattern associated with the breathing exercise

What is claimed is:

1. A stress management apparatus comprising:
a wearable device having one or more physiological sensors operable to be engaged with a body of a user;
one or more processors communicatively coupled with the wearable device, the one or more processors having a memory storing instructions when executed operable to:
detect one or more physiological indicators of stress;
suggest a stress intervention to the user;
monitor a compliance with the stress intervention; and
track a reduction of the one or more physiological indicators of stress.

2. The stress management apparatus of claim 1, wherein the one or more physiological sensors are an electrodemal sensor (EDA), galvanic skin response (GSR) sensor, a photoplethysmography (PPG), an electrocardiogram (EKG), an inertial measurement sensor, an accelerometer, a gyroscope, a blood pressure sensor, a pulse oximetry (SpO₂) sensor, a respiratory rate monitor, a temperature sensor, a humidity sensor, an audio sensor, and combinations thereof

3. The stress management apparatus of claim 1, wherein the one or more physiological indicators of stress are a change of one or more of skin temperature, heart rate, heart rate variability, blood pulse volume, skin conductance, skin impedance, blood pressure, breathing rate, blood oxygenation, and/or perspiration.

4. The stress management apparatus of claim 1, wherein a stress intervention is a breathing exercise having a predetermined sequence of inhale and/or exhale patterns.

5. The stress management apparatus of claim 4, wherein monitoring a compliance with the stress intervention monitors an inhale and/or exhale pattern of the user compared with the inhale and/or exhale pattern associated with the breathing exercise.

6. The stress management apparatus of claim 1, further comprising repeat the stress intervention if the compliance with the stress intervention is below a predetermined threshold.

7. The stress management apparatus of claim 1, further comprising one or more context sensors, wherein the one or more context sensors are operable to detect one or more environmental elements of the user.

8. The stress management apparatus of claim 7, wherein the one or more environmental elements are ambient temperature, humidity, air quality, noise level, ultraviolet (UV) level, and/or location.

9. The stress management apparatus of claim 8, wherein the one or more physiological indicators of stress are determined relative to the one or more environmental elements.

10. The stress management apparatus of claim 1, further comprising determine if the one or more physiological indicators of stress has changed.

11. The stress management apparatus of claim 1, further comprising establish a baseline for the one or more physiological indicators of stress in an unstressed state for the user.

12. The stress management apparatus of claim 11, wherein the baseline is adjusted through continuous monitoring of the one or more physiological sensors.

13. A method of stress management, the method comprising:
detecting, via one or more physiological sensors, one or more physiological indicators of stress;
suggesting a stress intervention to a user;
monitor, via the one or more physiological sensors, compliance with the stress intervention; and
tracking, via the one or more physiological sensors, a reduction of the one or more physiological indicators of stress.

14. The method of stress management of claim 13, further comprising establishing, via the one or more physiological sensors, a baseline for the one or more physiological indicators of stress in an unstressed state for the user.

15. The method of stress management of claim 14, further comprising adjusting the baseline of the one or more physiological indicators of stress continuously.

16. The method of stress management of claim 13, further comprising repeating the stress intervention if the compliance with the stress intervention is below a predetermined threshold.

17. The method of stress management of claim 13, wherein the one or more physiological indicators of stress are determined relative to one or more environmental elements operably detected by one or more context sensors.

18. The method of stress management of claim 17, wherein the one or more environmental elements are ambient temperature, humidity, air quality, noise level, ultraviolet (UV) level, and/or location.

19. The method of stress management of claim 13, wherein a stress intervention is a breathing exercise having a predetermined sequence of inhale and/or exhale patterns.

20. The method of stress management of claim 19, wherein monitoring compliance with the stress intervention monitors an inhale and/or exhale pattern of the user compared with the inhale and/or exhale pattern associated with the breathing exercise.
